(19) United States
(12) Patent Application Publication
(10) Pub. No.: US 2007/0110681 A1
(43) Pub. Date: May 17, 2007

(54) FLAVOR-STABLE DENTAL DEVICES

(75) Inventors: Ira D. Hill, Austin, TX (US); Robert C. Lepple, Missouri City, TX (US); Dale G. Brown, Wharton, TX (US)

Correspondence Address: BANNER & WITCOFF, LTD., 28 STATE STREET, 28th FLOOR, BOSTON, MA, 02109-9601, US

(73) Assignee: Whitehill Oral Technologies, Inc., Stafford, TX (US)

(21) Appl. No.: 11/549,671

(22) Filed: Oct. 16, 2006

Related U.S. Application Data
(63) Continuation-in-part of application No. 11/349,042, filed on Feb. 7, 2006.
(63) Continuation-in-part of application No. 11/380,331, filed on Apr. 26, 2006.
(63) Continuation-in-part of application No. 10/005,902, filed on Dec. 4, 2001.
(63) Continuation-in-part of application No. 10/331,800, filed on Dec. 30, 2002.
(63) Continuation-in-part of application No. 10/334,089, filed on Dec. 30, 2002, now Pat. No. 7,017,591.
(63) Continuation-in-part of application No. 11/118,911, filed on Apr. 29, 2005.
(63) Continuation-in-part of application No. 11/549,668, filed on Oct. 16, 2006.
(63) Continuation-in-part of application No. 11/549,670, filed on Oct. 16, 2006.
(60) Provisional application No. 60/745,704, filed on Apr. 26, 2006.

(51) Int. Cl. A61K 8/89; A61K 8/31

(52) U.S. Cl. 424/49

(57) ABSTRACT

Flavor-stable, dental devices are coated with a flavor-absorbing, saliva soluble coating and packaged in unit-dose packaging with a flavor-compatible wrapping material, wherein said unit-doses are contained in a flavor-sealed dispenser that has been purged with an inert gas and fitted with a volatile flavor reservoir, prior to sealing, whereby said flavor reservoir releases flavor that is absorbed by the saliva soluble coating on said dental device.

### CROSS-REFERENCE TO RELATED APPLICATIONS

This application is a continuation-in-part of the following cop ending applications: U.S. patent application Ser. No. 11/349,042, filed Feb. 7, 2006 entitled: “Sialagogue Coatings for Interproximal Devices”; “Methods for Coating Dental Devices with Sialagogue Emulsions, Ser. No. 11/380,331, filed Apr. 26, 2006; Methods for Coating Dental Devices with Dry-to-the-Touch Saliva Soluble Flavors, Ser. No. 60/745,704, filed Apr. 26, 2006 (now abandoned); U.S. patent application Ser. No. 10/005,902, filed Dec. 4, 2001 entitled “Biofilm Therapy Process and Elements”; U.S. patent application Ser. No. 10/331,800, filed Dec. 30, 2002, entitled, “Coated Micromesh Dental Devices Overcoated with Imbedded Particulate”; U.S. patent application, Ser. No. 11/118,911, filed Apr. 29, 2005, entitled, “Coated Monofilament Oriented HDPE Dental Tapes”; U.S. Pat. No. 7,017,591, entitled, “Particulate Coated Monofilament Devices”; U.S. Pat. Application, Serial No. 11/______, filed 16 Oct. 2006 (Attorney Docket No. 004526.00071), entitled, “Clean Perception Oral Products”; and U.S. Pat. Application, Serial No. 11/______, filed 16 Oct. 2006 (Attorney Docket No. 004526.00074), entitled, “Coated Dental Devices with Dry-to-the-Touch, Flavor-Absorbing, Saliva Soluble Coatings and Methods for Manufacturing”. The disclosures of these applications are hereby incorporated herein by reference.

### FIELD OF INVENTION

Traditionally, most dental devices, including dental flosses and dental tapes, have been coated with various coatings which can contain various types of flavors. Some of these are described in the following U.S. Pat. Nos.:

U.S. Pat. Nos.:

35,439; 428,033; 1,105,739; 1,336,272; 1,138,479;
1,285,988; 1,441,681; 1,471,987; 1,149,376; 1,627,963;
1,633,336; 1,716,035; 1,839,483; 1,839,486; 1,936,456;
1,943,856; and 1,989,895
2,004,957; 2,024,146; 2,027,535; 2,031,233; 2,035,267;
2,054,742; 2,069,157; 2,089,845; 2,124,971; 2,154,168;
2,224,489; 2,381,142; 2,464,755; 2,542,518; 2,554,464;
2,667,443; 2,677,700; 2,700,636; 2,748,781; 2,772,205;
2,778,045; 2,806,814; 2,896,639; 2,941,926; and 2,954,587;
3,137,632; 3,153,418; 3,164,524; 3,214,899; 3,219,527;
3,228,845; 3,254,714; 3,307,628; 3,330,732; 3,339,546;
3,417,179; 3,427;380; 3,427,381; 3,429,963; 3,429,964;
3,431,339; 3,433,780; 3,475,533; 3,491,776; 3,497,006;
3,506,070; 3,507,955; 3,574,824; 3,624,120; 3,629,468;
3,639,563; 3,651,207; 3,662,930; 3,664,915; 3,678,154;
3,693,851; 3,698,392; 3,699,979; 3,729,553; 3,737,533;
3,744,499; 3,771,536; 3,772,431; 3,789,858; 3,800,812;
3,830,246; 3,837,351; 3,838,702; 3,849,185; 3,864,472;
3,887,701; 3,888,976; 3,897,795; 3,897,796; 3,907,991;
3,911,099; 3,911,766; 3,927,201; 3,927,202; 3,928,618;
3,929,988; 3,942,539; 3,943,949; 3,947,570; 3,953,566;
3,956,480; 3,957,964; 3,962,153; 3,964,164; and 3,975,294;
4,008,727; 4,020,558; 4,022,880; 4,024,871; 4,029,113;
4,033,365; 4,034,771; 4,069,312; 4,071,614; 4,082,841;
4,096,2271 4,100,269; 4,110,429; 4,130,636; 4,132,770;
4,138,477; 4,142,538; 4,143,126; 4,146,499; 4,150,151;
4,151,851; 4,152,416; 4,152,418; 4,158,815; 4,159,619;
4,175,326; 4,187,390; 4,205,061; 4,215,478; 4,224,307;
4,224,308; 4,244,931; 4,247,526; 4,251,507; 4,256,806;
4,265,258; 4,277,297; 4,304,766; 4,329,333; 4,338,275;
4,339,429; 4,343,785; 4,353,890; 4,362,639; 4,367,759;
4,370,314; 4,385,093; 4,414,990; 4,420,472; 4,446,157;
4,450,849; 4,462,136; 4,465,661; 4,465,663; 4,476,107;
4,478,665; 4,510,127; 4,511,563; 4,525,342; 4,537,778;
4,548,219; 4,568,535; 4,569,837; 4,610,872; 4,611,309;
4,612,191; 4,620,878; 4,627,975; 4,638,823; 4,657,758;
4,661,341; 4,666,708; 4,764,377; 4,774,077; 4,776,358;
4,795,421; 4,832,063; 4,892,736; 4,894,220; 4,902,497;
4,908,247; 4,911,927; 4,933,182; 4,942,034; 4,947,880;
4,950,479; 4,974,614; 4,974,615; 4,981,693; 4,986,288;
4,996,011; and 4,998,011;
5,002,714; 5,009,881; 5,021,267; 5,032,387; 5,033,365;
5,033,488; 5,038,805; 5,057,306; 5,057,307; 5,057,308;
5,057,309; 5,057,310; 5,078,988; 5,091,133; 5,094,255;
5,098,711; 5,106,555; 5,106,558; 5,115,002; 5,129,824;
5,160,561; 5,165,713; 5,165,913; 5,183,063; 5,200,129;
5,209;251; 5,219,572; 5,220,932; 5,226,435; 5,284,169;
5,284,648; 5,289,836; 5,311,889; 5,311;890; 5,312,618;
5,316,028; 5,380,530; 5,353,820; 5,357,990; 5,423,337;
5,433,226; 5,479,952; 5,503,842; 5,505,216; 5,518,012;
5,520,351; 5,526,831; 5,538,667; 5,557,900; 5,558,452;
5,558,901; 5,560,377; 5,560,921; 5,561,959; 5,573,850;
5,578,373; 5,582,194; 5,598,373; 5,609,170; 5,616,315;
5,645,841; 5,651,959; 5,665,374; 5,693,708; 5,695,708;
5,697,390; 5,702,657; 5,711,935; 5,718,251; 5,723,388;
5,755,243; 5,760,117; 5,765,576; 5,787,758; 5,830,495;
5,845,652; 5,848,600; 5,865,197; 5,875,798; 5,884,639;
5,904,152; 5,911,228; 5,918,609; 5,937,874; 5,962,572;
5,967,153; 5,967,154; 5,988,444; and 5,998,431;
6,003,525; 6,016,816; 6,017,480; 6,027,192; 6,027,592;
6,027;593; 6,080,481; 6,083,208; 6,148,830; 6,161,555;
6,192,896; 6,198,830; 6,303,063; 6,371,133; 6,545,077;
6,575,176; 6,591,844; 6,604,534; 6,609,527; 6,884,309;
6,907,889; and 6,916,880;
7,017,591; 7,025,986; 7,011,099; 7,025,986; 7,060,354

U.S. patent application Nos.:

Ser. No. 10/005,902, filed Dec. 4, 2001, entitled, “Biofilm Therapy Process and Elements,” now U.S. Pat. Publication No. 2003-0035779 A1;

Ser. No. 11/196,827, filed Aug. 3, 2005, entitled “Biofilm Therapy Interproximal Devices,” now U.S. Pat. Publication No. 2006-0034782 A1;

Ser. No. 11/331,513, filed Jan. 13, 2006, entitled: “Particulate Coated Monofilament Devices,” now U.S. Pat. Publication No. 2006-0112968 A1;

Ser. No. 10/331,800, filed Dec. 30, 2002, entitled “Coated Micromesh Dental Devices Overcoated with Imbedded Particulate,” now U.S. Pat. Publication No. 2003-0168077 A1;

Ser. No. 10/331,795, filed Dec. 30, 2002, entitled “Coated Multifilament Dental Devices Overcoated with Imbedded Particulate,” now U.S. Pat. Publication No. 2004-0123877 A1;

Ser. No. 11/349,042, filed Feb. 7, 2006 entitled “Sialagogue Coatings for Interproximal Devices,” now U.S. Pat. Publication No. 2006-0177384 A1;

Ser. No. 11/380,331, filed Apr. 26, 2006, entitled “Methods for Coating Dental Devices with Sialagogue Emulsions,” now U.S. Pat. Publication No. 2006-0201531 A1;

Ser. No. 11/118,911, filed Apr. 29, 2005, entitled, “Coated Monofilament Oriented HDPE Dental Tapes,” now U.S. Pat. Publication No. 2006______, A1;

Ser. No. 60/745,704, filed Apr. 26, 2006, entitled: “Methods for Coating Dental Devices with Dry-to-the-Touch Saliva Soluble Flavors”;

Ser. No. 11/______, filed 16 Oct. 2006, (Attorney Docket No. 004526.00071), entitled, “Clean Perception Oral Products; and

Ser. No. 11/______, filed 16 Oct. 2006, (Attorney Docket No. 004526.00074), entitled, “Methods for Coating Dental Devices with Dry-to-the-Touch Saliva Soluble Coatings”.

### BACKGROUND OF THE INVENTION

At present, all commercial, “flavored,” coated, dental devices that are dispensed by means of a bobbin from a plastic dispenser experience flavor degradation. Some of these have been reviewed for flavor degradation, including olfactory and changes in taste. The results of this review are detailed in Tables 1 through 8 below. Tables 1 through 8 report on the olfactory and taste stability of various commercial, flavored, multifilament dental devices and monofilament dental devices maintained under various conditions over a period of five (5) through thirty-five (35) days.

TABLE 1
Branded Coated Multifilament Dental Flosses

| | | PANEL EVALUATION (For each brand of | | | |
|---|---|---|---|---|---|
| | | dispensers, ⅓ were stored at 40° C. Dry, | | | |
| | | ⅓ were stored at 40° C. 75% humidity, and the remaining | | | |
| | | were stored at 5° C. as Controls. The dispensers were compared | | | |
| | | to Controls and evaluated for Odor and Taste intensity, and the | | | |
| | | intensity was expressed as percent relative to Control) | | | |
| | | Comparative | | Comparative | |
| | | Olfactory Stability | | Taste Stability | |
| | Duration | (Compare to Control) | | (Compare to Control) | |
| | of test | | 40° C. 75% | | 40° C. 75% |
| BRAND | (in days) | 40° C. Dry | humidity | 40° C. Dry | humidity |
| J&J REACH ® | 5 | Intensity = 97 | Intensity = 98 | Intensity = 98 | Intensity = 98 |
| Clean Paste ™ | | | | | |
| Original | | | | | |
| J&J REACH ® | 5 | Intensity = 97 | Intensity = 97 | Intensity = 95 | Intensity = 97 |
| Clean Paste ™ | | | | | |
| Tartar | | | | | |
| J&J REACH ® | 5 | Intensity = 95 | Intensity = 95 | Intensity = 97 | Intensity = 97 |
| GGC with | | | | | |
| Fluoride | | | | | |
| J&J REACH ® | 5 | Intensity = 93 | Intensity = 93 | Intensity = 92 | Intensity = 88 |
| Clean Burst | | | | | |

TABLE 2
Branded Coated Monofilament Dental Tapes

| | | PANEL EVALUATION (For each brand of dispensers, | | | |
|---|---|---|---|---|---|
| | | ⅓ were stored at 40° C. Dry, ⅓ were stored at 40° C. | | | |
| | | 75% humidity, and the remaining were stored at 5° C. | | | |
| | | as Controls. The dispensers were compared to | | | |
| | | Controls and evaluated for Odor and Taste intensity, and the | | | |
| | | intensity was expressed as percent relative to Control) | | | |
| | | Comparative | | Comparative | |
| | | Olfactory Stability | | Taste Stability | |
| | Duration | (Compare to Control) | | (Compare to Control) | |
| | of test | | 40° C. 75% | | 40° C. 75% |
| BRAND | (in days) | 40° C. Dry | humidity | 40° C. Dry | humidity |
| J&J REACH ® | 5 | Intensity = 95 | Intensity = 98 | Intensity = 95 | Intensity = 98 |
| Clean Paste ™ | | | | | |
| Tight Teeth | | | | | |
| Glide - Deep | 2 | Intensity = 95 | Intensity = 98 | Intensity = 95 | Intensity = 98 |
| Clean | | | | | |
| Oral B - Satin | 2 | Intensity = 85 | Intensity = 87 | Intensity = 87 | Intensity = 87 |
| Tape | | | | | |

TABLE 3
Branded Coated Multifilament Dental Flosses

| | | PANEL EVALUATION (For each brand of dispensers, | | | |
|---|---|---|---|---|---|
| | | ⅓ were stored at 40° C. Dry, ⅓ were stored at 40° C. | | | |
| | | 75% humidity, and the remaining were stored at 5° C. as | | | |
| | | Controls. The dispensers were compared to Controls and | | | |
| | | evaluated for Odor and Taste intensity, and the intensity | | | |
| | | was expressed as percent relative to Control) | | | |
| | | Comparative | | Comparative | |
| | | Olfactory Stability | | Taste Stability | |
| | Duration | (Compare to Control) | | (Compare to Control) | |
| | of test | | 40° C. 75% | | 40° C. 75% |
| BRAND | (in days) | 40° C. Dry | humidity | 40° C. Dry | humidity |
| J&J REACH ® | 20 | Intensity = 82 | Intensity = 84 | Intensity = 84 | Intensity = 82 |
| Clean Paste ™ | | | | | |
| Original | | | | | |
| J&J REACH ® | 20 | Intensity = 78 | Intensity = 74 | Intensity = 83 | Intensity = 78 |
| Clean Paste ™ | | | | | |
| Tartar | | | | | |
| J&J REACH ® | 20 | Intensity = 77 | Intensity = 83 | Intensity = 76 | Intensity = 83 |
| GGC with | | | | | |
| Fluoride | | | | | |
| J&J REACH ® | 20 | Intensity = 82 | Intensity = 82 | Intensity = 82 | Intensity = 82 |
| Clean Burst | | | | | |

TABLE 4
Branded Coated Monofilament Dental Tapes

| | | PANEL EVALUATION (For each brand of dispensers, | | | |
|---|---|---|---|---|---|
| | | ⅓ were stored at 40° C. Dry, ⅓ were stored at 40° C. | | | |
| | | 75% humidity, and the remaining were stored at 5° C. | | | |
| | | as Controls. The dispensers were compared to Controls | | | |
| | | and evaluated for Odor and Taste intensity, and the intensity | | | |
| | | was expressed as percent relative to Control) | | | |
| | | Comparative | | Comparative | |
| | | Olfactory Stability | | Taste Stability | |
| | Duration | (Compare to Control) | | (Compare to Control) | |
| | of test | | 40° C. 75% | | 40° C. 75% |
| BRAND | (in days) | 40° C. Dry | humidity | 40° C. Dry | humidity |
| J&J REACH ® | 20 | Intensity = 85 | Intensity = 90 | Intensity = 88 | Intensity = 93 |
| Clean Paste ™ | | | | | |
| Tight Teeth | | | | | |
| Glide —Deep | 17 | Intensity = 83 | Intensity = 81 | Intensity = 84 | Intensity = 79 |
| Clean | | | | | |
| Oral B —Satin | 17 | Intensity = 14⁽¹⁾ | Intensity = 68 | Intensity = 15 | Intensity = 67 |
| Tape | | | | | |

⁽¹⁾Segment of floss tested was not coated.

TABLE 5
Branded Coated Multifilament Dental Flosses

| | | PANEL EVALUATION (For each brand of dispensers, | | | |
|---|---|---|---|---|---|
| | | ⅓ were stored at 40° C. Dry, ⅓ were stored at 40° C. | | | |
| | | 75% humidity, and the remaining were stored at 5° C. as | | | |
| | | Controls. The dispensers were compared to Controls and | | | |
| | | evaluated for Odor and Taste intensity, and the intensity | | | |
| | | was expressed as percent relative to Control) | | | |
| | | Comparative | | Comparative | |
| | | Olfactory Stability | | Taste Stability | |
| | Duration | (Compare to Control) | | (Compare to Control) | |
| | of test | | 40° C. 75% | | 40° C. 75% |
| BRAND | (in days) | 40° C. Dry | humidity | 40° C. Dry | humidity |
| J&J REACH ® | 28 | Intensity = 69 | Intensity = 74 | Intensity = 73 | Intensity = 78 |
| Clean Paste ™ | | | | | |
| Original | | | | | |
| J&J REACH ® | 28 | Intensity = 65 | Intensity = 71 | Intensity = 65 | Intensity = 78 |
| Clean Paste ™ | | | | | |
| Tartar | | | | | |
| J&J REACH ® | 28 | Intensity = 53 | Intensity = 74 | Intensity = 55 | Intensity = 84 |
| GGC with | | | | | |
| Fluoride | | | | | |
| J&J REACH ® | 28 | Intensity = 79 | Intensity = 79 | Intensity = 81 | Intensity = 74 |
| Clean Burst | | | | | |

TABLE 6
Branded Coated Monofilament Dental Tapes

| | | PANEL EVALUATION (For each brand of dispensers, | | | |
|---|---|---|---|---|---|
| | | ⅓ were stored at 40° C. Dry, ⅓ were stored at 40° C. | | | |
| | | 75% humidity, and the remaining were stored at 5° C. as | | | |
| | | Controls. The dispensers were compared to Controls and | | | |
| | | evaluated for Odor and Taste intensity, and the intensity | | | |
| | | was expressed as percent relative to Control) | | | |
| | | Comparative | | Comparative | |
| | | Olfactory Stability | | Taste Stability | |
| | Duration | (Compare to Control) | | (Compare to Control) | |
| | of test | | 40° C. 75% | | 40° C. 75% |
| BRAND | (in days) | 40° C. Dry | humidity | 40° C. Dry | humidity |
| J&J REACH ® | 28 | Intensity = 84 | Intensity = 76 | Intensity = 88 | Intensity = 79 |
| Clean Paste ™ | | | | | |
| Tight Teeth | | | | | |
| Glide - Deep | 25 | Intensity = 74 | Intensity = 79 | Intensity = 74 | Intensity = 79 |
| Clean | | | | | |
| Oral B - Satin | 25 | Intensity = 50 | Intensity = 60 | Intensity = 49 | Intensity = 44 |
| Tape | | | | | |

TABLE 7
Branded Coated Multifilament Dental Flosses

| | | PANEL EVALUATION (For each brand of dispensers, | | | |
|---|---|---|---|---|---|
| | | ⅓ were stored at 40° C. Dry, ⅓ were stored at 40° C. | | | |
| | | 75% humidity, and the remaining were stored at 5° C. as | | | |
| | | Controls. The dispensers were compared to Controls | | | |
| | | and evaluated for Odor and Taste intensity, and the intensity | | | |
| | | was expressed as percent relative to Control) | | | |
| | | Comparative | | Comparative | |
| | | Olfactory Stability | | Taste Stability | |
| | Duration | (Compare to Control) | | (Compare to Control) | |
| | of test | | 40° C. 75% | | 40° C. 75% |
| BRAND | (in days) | 40° C. Dry | humidity | 40° C. Dry | humidity |
| J&J REACH ® | 35 | Intensity = 71 | Intensity = 77 | Intensity = 81 | Intensity = 84 |
| Clean Paste ™ | | | | | |
| Original | | | | | |
| J&J REACH ® | 35 | Intensity = 56 | Intensity = 59 | Intensity = 64 | Intensity = 61 |
| Clean Paste ™ | | | | | |
| Tartar | | | | | |
| J&J REACH ® | 35 | Intensity = 65 | Intensity = 66 | Intensity = 63 | Intensity = 68 |
| GGC with | | | | | |
| Fluoride | | | | | |
| J&J REACH ® | 35 | Intensity = 80 | Intensity = 76 | Intensity = 77 | Intensity = 67 |
| Clean Burst | | | | | |

TABLE 8
Branded Coated Monofilament Dental Tapes

| | | PANEL EVALUATION (For each brand of dispensers, | | | |
|---|---|---|---|---|---|
| | | ⅓ were stored at 40° C. Dry, ⅓ were stored at 40° C. | | | |
| | | 75% humidity, and the remaining were stored at 5° C. as | | | |
| | | Controls. The dispensers were compared to Controls and | | | |
| | | evaluated for Odor and Taste intensity, and the intensity | | | |
| | | was expressed as percent relative to Control) | | | |
| | | Comparative | | Comparative | |
| | | Olfactory Stability | | Taste Stability | |
| | Duration | (Compare to Control) | | (Compare to Control) | |
| | of test | | 40° C. 75% | | 40° C. 75% |
| BRAND | (in days) | 40° C. Dry | humidity | 40° C. Dry | humidity |
| J&J REACH ® | 35 | Intensity = 77 | Intensity = 85 | Intensity = 81 | Intensity = 86 |
| Clean Paste ™ | | | | | |
| Tight Teeth | | | | | |
| Glide - Deep | 32 | Intensity = 81 | Intensity = 76 | Intensity = 78 | Intensity = 68 |
| Clean | | | | | |
| Oral B - Satin | 32 | Intensity = 48 | Intensity = 63 | Intensity = 53 | Intensity = 38 |
| Tape | | | | | |

It is evident from the observations reported in Tables 1 through 8 that various commercial, coated dental devices containing a wide range of flavors at several levels, when dispensed from traditional plastic dispensers, using bobbin-wound dispensing means, consistently undergo flavor loss, flavor change and/or flavor degradation, such that, these devices generally lack the beneficial, consumer-friendly, flavor attributes that were initially sought when the flavored coating was applied. In general, various attempts to minimize such flavor change, including resorting to powdered, encapsulated, or other processed flavors, or using less volatile flavors, etc., have not been successful.

The influence of high-flavor-impact on consumer response to various dental devices is well documented and includes shifting consumer preferences dramatically towards the higher impact flavored devices. Some of the coated devices listed in Tables 1 through 8 above, contain upwards of about 8% by weight flavor. This represents a major cost-of-goods investment, which eventually falls short in consumer appeal, as these coated devices, over time, undergo continuing flavor loss/flavor degradation, prior to and after purchase.

Most of the flavors used in the coated devices listed in Tables 1 through 8 above are eventually lost and/or degraded due to various conditions encountered by these flavors prior to use and after purchase, including being:
(a) adsorbed by the plastic dispenser, and/or surrounding packaging materials,
(b) modified through oxidation, and/or
(c) volatilized and dissipated from the coating throughout the bobbin when exposed to elevated temperatures during storage.

Additionally, under certain conditions, certain fractions of the flavor are selectively adsorbed by the various dispensing and packaging components associated with the device resulting in a “fractionation” of the flavor, which also results in undesirable alterations of the flavor perception.

Clearly, there is a need for flavor-stable dental devices that provide the consumer flavored dental devices with high-flavor-impact free from:
Flavor fractionation,
Flavor degradation,
Flavor absorption by packaging/dispensing components,
Flavor oxidation, and/or
Flavor volatilization.

### OBJECTS OF THE INVENTION

An object of the invention is to provide flavor-stable dental devices that are free from:
Flavor fractionation,
Flavor degradation,
Flavor absorption by packaging/dispensing components,
Flavor oxidation, and/or
Flavor volatilization.

A further object of the invention is to provide a method for improving long-term flavor stability of coated dental devices.

Another object of the invention is to provide an improved coated dental device dispensing means that delivers long-term flavor stability.

Yet another object of the invention is to provide an alternative flavor-stable dispensing means to replace all forms of bobbin-dispensed dental devices.

Still another object of the invention is to provide an improved dental device dispensing means that provides long-term stability to various active ingredients included in the device coating.

Yet another object of the invention is to improve the flavor stability of all commercial dental devices.

Another object of the invention is to provide an improved method for packaging coated dental devices, where the primary flavor is added to said devices after coating.

A further object of the invention is to provide an improved method for unit-dose packaging of coated dental devices that minimizes coating loss, while not interfering nor modifying post-flavor-transfer to said coated device.

Still another object of the invention is to provide an improved method for unit-dose packaging of coated dental devices that obviates the need for heat and/or gluing.

A further object of the invention is to provide stable packaging for normally unstable ingredients contained in dental device coatings, where the normally unstable ingredients remain stable.

These and other objects of the invention are achievable and understandable by one skilled in the art after reviewing the specification, examples and claims set out below.

### SUMMARY OF THE INVENTION

The present invention is directed to flavor-stable dental devices which are coated with a flavor-absorbing, saliva soluble coating. These coated dental devices are packaged in unit-doses with a wrapping material that allows volatile flavors to pass through the wrapping material in order to be absorbed by the coating, while neither interfering with nor altering the volatile flavor. These wrapped, unit-doses of coated dental devices are subsequently contained under flavor-sealed packaging that is:
(a) purged with an inert gas prior to sealing, and
(b) provided with a flavor reservoir containing a volatile flavor that is absorbable by the dental device coating.

These flavor-stable, dental device, unit-doses not only feature extended flavor stability, but they also provide stability to various chemotherapeutic ingredients contained in the device coating that would otherwise be vulnerable to degradation upon being exposed to the elements when contained and dispensed from traditional bobbin dispensers used for dental devices.

For example, certain chemotherapeutic ingredients contained in dental device coatings, that are effective when delivered to specific interproximal sites tend to be unstable when exposed to the elements. These include: stannous fluoride, baking soda, various peroxide salts, hexametaphosphate, complexing ingredients, certain antimicrobials, antibiotics, enzymes, NSAIDS, MMP inhibitors, vitamins, nutraceuticals, etc. All of these foregoing can be included in various dental device coatings and remain stable and effective under the unit-dose packaging and flavor-sealed packaging provisions of the present invention until they are delivered to specific interproximal sites by flossing.

Thus, one aspect of the present invention is directed a flavor-stable, dental device having a flavor-absorbing, saliva soluble coating, covered with a flavor-compatible, unit-dose packaging and contained in a flavor-sealed dispenser that has been purged with an inert gas and fitted with a volatile flavor reservoir. Preferably, the primary source of flavor is added to said dental device after coating.

Preferably, the flavor-stable, dental device comprises a flavor-absorbing, saliva soluble coating that is selected from the group consisting of: surfactants, waxes, emulsions, film-forming agents, crystal-free coatings, lipowaxes, substantive coatings and combinations thereof.

Preferably, the flavor-stable, dental device comprises a saliva soluble coating that contains an emulsion comprising a polydimethylsiloxane dispersed in a surfactant. Alternatively or additionally, the flavor-stable, dental device comprises a saliva soluble coating that contains a wax. Alternatively or additionally, the flavor-stable, dental device comprises a saliva soluble coating that contains a lipowax. Alternatively or additionally, the flavor-stable, dental device comprises a saliva soluble coating that contains at least one sialagogue.

Preferably, the flavor-stable, dental device comprises a flavor-absorbing, saliva soluble coating selected from the group consisting of: nylon, multifilament dental floss; texturized nylon, multifilament dental floss; polytetrafluoroethylene, monofilament dental tape; high density polyethylene, monofilament dental tape; ultra high molecular weight, polyethylene, monofilament dental tape; and combinations thereof.

Preferably, the flavor-stable, dental device comprises a saliva soluble coating that further contains a chemotherapeutic ingredient selected from the group consisting of: stannous fluoride, baking soda, peroxide salts, hexametaphosphate, complexing ingredients, antimicrobials, antibiotics, enzymes, NSAIDs, MMP inhibitors, vitamins, nutraceuticals and combinations thereof.

Preferably, the flavor-stable, dental device comprises a saliva soluble coating that further contains the chemotherapeutic ingredient, baking soda. Additionally or alternatively, a preferred chemotherapeutic ingredient is hexametaphosphate.

Preferably, the flavor-stable, dental device comprises a flavor reservoir selected from the group consisting of: paper, blotter material, cigarette paper, tissue paper, ethylene vinyl acetate resins, natural resins and combinations thereof.

Another aspect of the present invention is directed to a method for improving the long-term flavor stability of dental devices comprising:
(a) coating said devices with flavor-absorbing coating,
(b) unit-dose packaging said coated dental devices with a flavor-compatible wrapping, and
(c) storing said coated unit-dose dental devices in a flavor-sealed package that has been purged with an inert gas and provided with a volatile flavor reservoir.

Preferably, the method for improving the long-term flavor stability of dental devices employs a flavor-absorbing coating that is selected from the group consisting of: surfactants, waxes, emulsions, film-forming agents, crystal-free coatings, lipowaxes, substantive coating and combinations thereof.

Preferably, the method for improving the long-term flavor stability of dental devices employs a dental device, having a flavor-absorbing, saliva soluble coating selected from the group consisting of: nylon, multifilament dental floss; texturized nylon, multifilament dental floss; polytetrafluoroethylene, monofilament dental tape; high density polyethylene, monofilament dental tape; ultra high molecular weight, polyethylene, monofilament dental tape; and combinations thereof.

Preferably, the method for improving the long-term flavor stability of dental devices employs a dental device, wherein the flavor-absorbing coating further comprises chemotherapeutic ingredients selected from the group consisting of stannous fluoride, baking soda, peroxide salts, hexametaphosphate, complexing ingredients, antimicrobials, antibiotics, enzymes, NSAIDs, MMP inhibitors, vitamins, nutraceuticals and combinations thereof. Preferably, the chemotherapeutic ingredient is baking soda. Alternatively or additionally, a preferred chemotherapeutic ingredient is hexametaphosphate.

Another aspect of the present invention is directed to a method for post-adding flavor to coated dental devices, comprising:
(a) coating said dental devices with a flavor-absorbing coating, that is substantially free from flavor,
(b) unit-dose packaging said coated dental devices with a flavor-compatible wrapping, and
(c) storing said coated, unit-dose, dental device in a flavor-sealed package that has been purged with an inert gas and provided with a flavor reservoir prior to sealing.

Another aspect of the present invention is directed to a method for improving the long-term stability of dental devices containing chemotherapeutic ingredients, comprising:
(a) coating said dental devices with a saliva soluble coating containing an active ingredient,
(b) unit-dose packaging said coated dental devices with a flavor-compatible wrapping, and
(c) storing said coated, unit-dose, dental devices in a hermetically-sealed package that has been purged with an inert gas prior to sealing.

Another aspect of the present invention is directed to a method for unit-dose packaging of coated dental devices, comprising wrapping said devices with a flavor-compatible, yet flavor-permeable paper that is physically secured around said coated devices in the absence of a flavor-altering means.

Another aspect of the present invention is the conversion of the dental devices described in Tables 1 through 8 (above), into unit-doses, covered with a flavor-compatible packaging and contained in a flavor-sealed dispenser that has been purged with an inert gas and fitted with a volatile flavor reservoir. Both the devices so converted and the method of the conversion are preferred embodiments of the present invention.

### BRIEF DESCRIPTION OF THE DRAWINGS

FIG. 1 schematically illustrates a straw wrapping machine suitable for wrapping flavor-adsorbable coated dental tapes and dental flosses of the invention with flavor-compatible paper.

FIGS. 2 through 5 schematically illustrate several shapes of wrapped, coated dental tapes of the invention, prior to placement of the various shapes of wrapped devices into a flavor-sealed package.

FIGS. 6 through 12 are a series of bar charts representing the olfactory and taste stability data for various commercial dental flosses and dental tapes described in Tables 1 through 8.

### DEFINITIONS

For purposes of describing the present invention, the following terms, as used throughout this specification are defined as set out below.

“Dental Devices” are defined as coated dental flosses and coated dental tapes, where the coatings are suitable for absorbing volatile flavors, and/or can be modified to adsorb volatile flavors, including but not limited to those described, suggested, taught and/or claimed in the following U.S. Pat. Nos. and U.S. patent applications Ser. Nos.:

U.S. Pat. Nos.:

35,439; 428,033; 1,105,739; 1,336,272; 1,138,479;
1,285,988; 1,441,681; 1,471,987; 1,149,376; 1,627,963;
1,633,336; 1,716,035; 1,839,483; 1,839,486; 1,936,456;
1,943,856; and 1,989,895
2,004,957; 2,024,146; 2,027,535; 2,031,233; 2,035,267;
2,054,742; 2,069,157; 2,089,845; 2,124,971; 2,154,168;
2,224,489; 2,381,142; 2,464,755; 2,542,518; 2,554,464;
2,667,443; 2,677,700; 2,700,636; 2,748,781; 2,772,205;
2,778,045; 2,806,814; 2,896,639; 2,941,926; and 2,954,587;
3,137,632; 3,153,418; 3,164,524; 3,214,899; 3,219,527;
3,228,845; 3,254,714; 3,307,628; 3,330,732; 3,339,546;
3,417,179; 3,427;380; 3,427,381; 3,429,963; 3,429,964;
3,431,339; 3,433,780; 3,475,533; 3,491,776; 3,497,006;
3,506,070; 3,507,955; 3,574,824; 3,624,120; 3,629,468;
3,639,563; 3,651,207; 3,662,930; 3,664,915; 3,678,154;
3,693,851; 3,698,392; 3,699,979; 3,729,553; 3,737,533;
3,744,499; 3,771,536; 3,772,431; 3,789,858; 3,800,812;
3,830,246; 3,837,351; 3,838,702; 3,849,185; 3,864,472;
3,887,701; 3,888,976; 3,897,795; 3,897,796; 3,907,991;
3,911,099; 3,911,766; 3,927,201; 3,927,202; 3,928,618;
3,929,988; 3,942,539; 3,943,949; 3,947,570; 3,953,566;
3,956,480; 3,957,964; 3,962,153; 3,964,164; and 3,975,294;
4,008,727; 4,020,558; 4,022,880; 4,024,871; 4,029,113;
4,033,365; 4,034,771; 4,069,312; 4,071,614; 4,082,841;
4,096,2271 4,100,269; 4,110,429; 4,130,636; 4,132,770;
4,138,477; 4,142,538; 4,143,126; 4,146,499; 4,150,151;
4,151,851; 4,152,416; 4,152,418; 4,158,815; 4,159,619;
4,175,326; 4,187,390; 4,205,061; 4,215,478; 4,224,307;
4,224,308; 4,244,931; 4,247,526; 4,251,507; 4,256,806;
4,265,258; 4,277,297; 4,304,766; 4,329,333; 4,338,275;
4,339,429; 4,343,785; 4,353,890; 4,362,639; 4,367,759;
4,370,314; 4,385,093; 4,414,990; 4,420,472; 4,446,157;
4,450,849; 4,462,136; 4,465,661; 4,465,663; 4,476,107;
4,478,665; 4,510,127; 4,511,563; 4,525,342; 4,537,778;
4,548,219; 4,568,535; 4,569,837; 4,610,872; 4,611,309;
4,612,191; 4,620,878; 4,627,975; 4,638,823; 4,657,758;
4,661,341; 4,666,708; 4,764,377; 4,774,077; 4,776,358;
4,795,421; 4,832,063; 4,892,736; 4,894,220; 4,902,497;
4,908,247; 4,911,927; 4,933,182; 4,942,034; 4,947,880;
4,950,479; 4,974,614; 4,974,615; 4,981,693; 4,986,288;
4,996,011; and 4,998,011;
5,002,714; 5,009,881; 5,021,267; 5,032,387; 5,033,365;
5,033,488; 5,038,805; 5,057,306; 5,057,307; 5,057,308;
5,057,309; 5,057,310; 5,078,988; 5,091,133; 5,094,255;
5,098,711; 5,106,555; 5,106,558; 5,115,002; 5,129,824;
5,160,561; 5,165,713; 5,165,913; 5,183,063; 5,200,129;
5,209;251; 5,219,572; 5,220,932; 5,226,435; 5,284,169;
5,284,648; 5,289,836; 5,311,889; 5,311;890; 5,312,618;
5,316,028; 5,380,530; 5,353,820; 5,357,990; 5,423,337;
5,433,226; 5,479,952; 5,503,842; 5,505,216; 5,518,012;
5,520,351; 5,526,831; 5,538,667; 5,557,900; 5,558,452;
5,558,901; 5,560,377; 5,560,921; 5,561,959; 5,573,850;
5,578,373; 5,582,194; 5,598,373; 5,609,170; 5,616,315;
5,645,841; 5,651,959; 5,665,374; 5,693,708; 5,695,708;
5,697,390; 5,702,657; 5,711,935; 5,718,251; 5,723,388;
5,755,243; 5,760,117; 5,765,576; 5,787,758; 5,830,495;
5,845,652; 5,848,600; 5,865,197; 5,875,798; 5,884,639;
5,904,152; 5,911,228; 5,918,609; 5,937,874; 5,962,572;
5,967,153; 5,967,154; 5,988,444; and 5,998,431;
6,003,525; 6,016,816; 6,017,480; 6,027,192; 6,027,592;
6,027;593; 6,080,481; 6,083,208; 6,148,830; 6,161,555;
6,192,896; 6,198,830; 6,303,063; 6,371,133; 6,545,077;
6,575,176; 6,591,844; 6,604,534; 6,609,527; 6,884,309;
6,907,889; and 6,916,880;
7,017,591; 7,025,986; 7,011,099; 7,025,986; and 7,060,354

U.S. patent application Ser. Nos.:

Ser. No. 10/005,902, filed Dec. 4, 2001, entitled, “Biofilm Therapy Process and Elements,” now U.S. Pat. Publication No. 2003-0035779 A1;

Ser. No. 11/196,827, filed Aug. 3, 2005, entitled “Biofilm Therapy Interproximal Devices,” now U.S. Pat. Publication No. 2006-0034782 A1;

Ser. No. 11/331,513, filed Jan. 13, 2006, entitled: “Particulate Coated Monofilament Devices,” now U.S. Pat. Publication No. 2006-0112968 A1;

Ser. No. 10/331,800, filed Dec. 30, 2002, entitled “Coated Micromesh Dental Devices Overcoated with Imbedded Particulate,” now U.S. Pat. Publication No. 2003-0168077 A1;

Ser. No. 10/331,795, filed Dec. 30, 2002, entitled “Coated Multifilament Dental Devices Overcoated with Imbedded Particulate,” now U.S. Pat. Publication No. 2004-0123877 A1;

Ser. No. 11/349,042, filed Feb. 7, 2006 entitled “Sialagogue Coatings for Interproximal Devices,” now U.S. Pat. Publication No. 2006-0177384 A1;

Ser. No. 11/380,331, filed Apr. 26, 2006, entitled “Methods for Coating Dental Devices with Sialagogue Emulsions,” now U.S. Pat. Publication No. 2006-0201531 A1;

Ser. No. 11/118,911, filed Apr. 29, 2005, entitled, “Coated Monofilament Oriented HDPE Dental Tapes,” now U.S. Pat. Publication No. 2006______ A1;

Ser. No. 60/745,704, filed Apr. 26, 2006, entitled: “Methods for Coating Dental Devices with Dry-to-the-Touch Saliva Soluble Flavors”;

Serial No. 11/______, filed 16 Oct. 2006, (Attorney Docket No. 004526.00071), entitled, “Clean Perception Oral Products; and

Serial No. 11/______, filed 16 Oct. 2006, (Attorney Docket No. 004526.00074), entitled, “Methods for Coating Dental Devices with Dry-to-the-Touch Saliva Soluble Coatings”.

All of the foregoing U.S. Pat. Nos. and Patent Applications are incorporated herein by reference.

“Flavor-absorbing, saliva soluble coatings” are defined as those saliva soluble or saliva dispersible substances suitable for coating dental flosses and dental tapes that are capable of absorbing volatile flavors. These coatings are released from such devices during flossing. Suitable coating substances include: surfactants, waxes, lipowaxes, emulsions, film-forming agents, crystal-free coatings, and substantive coatings, which are suitable for use in the oral cavity. All of these coating substances are capable of absorbing volatile flavors when applied to dental devices that are subsequently contained under flavor-sealed conditions and provided with a flavor reservoir that releases various absorbed volatile flavors into the purged head-space of the flavor-sealed package.

“Flavor-compatible wrapping material for dental device, unit-dose packaging” is defined as those materials suitable for wrapping “flossing quantities” of coated dental devices into unit-doses. Generally, between about 18 to 22 inches of a dental device are used for each flossing session. Such flavor-compatible wrapping materials allow volatile flavors to pass through unaltered to be absorbed by the coatings on the dental devices. Not only are these wrapping materials compatible with the transfer of volatile flavors from the flavor reservoir to the device coatings, these wrapping materials are also free from glue, coatings, etc., and rely on other “physical attachment means” that do not adsorb and/or interfere with the adsorption of volatile flavors by the coated dental devices. Suitable flavor-compatible wrapping materials include various paper-based materials approved for food contact, such as used in various unit-dose food packages including beverage straw wrappers.

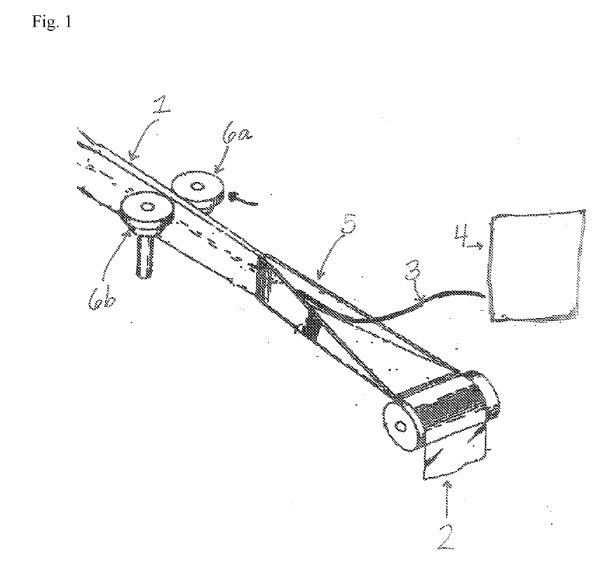

Traditionally, such food unit-dose wrappings are glue-free and use various physical attachment means to complete the wrapping such as crimping. See U.S. Pat. Nos. 2,280,405; 3,477,191; and 6,212,860. Paper wrapping machines for beverage straws are available from Conair such as the SWCB machine. See FIG. 1 of the Drawings. Generally, these paper “food wrappings” are printed with food-approved printing materials. A particularly preferred commercial paper is supplied by Clifford Paper, Upper Saddle River, N.J. Paper materials useful for wrapping these items are referred to as “FDA approved for food contact.” Other sources for suitable “wrapping material” include cigarette paper, tissue paper and the like.

A “flavor-sealed dispenser” is defined as a hermetically-sealed package that maintains the unit-dose, wrapped, coated dental device in an environment that supports absorption of volatile flavors by the coating on the device, where such absorption is carried out free from interference and/or alteration. The flavor-sealed dispenser is preferably purged with an inert gas and provided with a volatile flavor reservoir, prior to sealing. Suitable materials for the flavor-sealed dispenser include various flavor-sealable packaging materials that are suitable for containing volatile flavors without: interfering, altering and/or absorbing such volatile flavors. A particularly preferred packaging material for the flavor-sealed dispenser is produced from various Barex® resins. See discussion and Examples below.

“Inert gases suitable for purging the flavor-sealed dispenser” are defined as those gaseous materials that can be used to purge the flavor-sealed dispenser, in order to replace the air present with an inert gaseous material that does not interfere with nor degrade the volatile flavor to be introduced through the flavor reservoir, nor degrade various chemotherapeutic ingredients contained in the device coating. Suitable inert gases include: nitrogen, argon, neon, carbon dioxide, helium and xenon.

A “volatile flavor reservoir” is defined as the source for volatile flavors, contained in the flavor-sealed dispenser. These volatile flavors are absorbed by the flavor reservoir and released into the head space of the flavor-sealed dispenser, to be absorbed by the coatings on the unit-doses of dental devices. To insure stability of the volatile flavor in the head-space of the flavor-sealed dispenser, the flavor-sealed dispenser is purged with an inert gas prior to sealing. Suitable flavor reservoir materials include various absorbent substances such as blotter paper, tissue paper, natural resins, polymers, EVA resins, cotton, cellulose and other inert absorptive substances that can contain a wide range of volatile flavors which are held in the flavor reservoir to be released into the head space of the flavor-sealed dispenser for subsequent absorption by the dental device coatings. Suitable volatile flavors for use with the flavor reservoir include various: lemon, lime, mint, cinnamon, vanilla, peppermint, spearmint, tangerine, orange oil volatile notes and combinations thereof, as detailed in the Examples below. Preferably, these volatile flavors have flesh points from between about 116° and 119° F.

### DETAILED DESCRIPTION OF THE INVENTION

The flavor-stable dental devices of the invention include: (I) a flavor-absorbing, saliva soluble coating, (TI) unit-dose packaging with a flavor-compatible wrapping material and (ITT) A flavor-sealed dispenser for the unit-dose coated dental device, which is fitted with a flavor reservoir and purged with an inert gas. These are each discussed in detail below:

I. Flavor-Absorbing, Saliva Soluble Coatings:

The flavor-absorbing, saliva soluble, dental device coatings of the invention include a wide range of coating substances suitable for use in the oral cavity, including: surfactants, waxes, emulsions, film-forming agents, crystal-free coatings, lipowaxes and substantive coatings. Suitable waxes include synthetic and natural waxes such as carnauba, bees wax, paraffin, lanolin, candelilla, castor, ceresin, microcrystalline and polyethylene waxes.

Traditional wax coatings used on dental flosses and dental tapes are preferably modified by adding flavor-absorbing ingredients including surfactants, emulsions and film-formers. Microcrystalline, bees wax and emulsifying waxes are currently being used to coat dental floss and tape. Preferably, these waxes are emulsified with surfactants to form more effective flavor-adsorbable coatings.

Preferred flavor-absorbing saliva soluble coatings for use with the dental tapes and dental flosses of the invention include:
(a) those emulsion coatings described in the following U.S. Pat. Nos. 4,950,479; 5,032,387; 5,538,667; 5,561,959; and 5,665,374, which are hereby incorporated by reference,
(b) various dental device coatings, such as described in U.S. Pat. Nos. 5,908,039; 6,080,495; 4,029,113; 2,667,443; 3,943,949; 6,026,829; 5,967,155 and 5,967,153, which are hereby incorporated by reference,
(c) those substantive saliva soluble coatings described and claimed in U.S. Pat. Nos. 6,907,889; 6,609,527; 6,916,880 and 6,545,077, which are hereby incorporated by reference, and
(d) those coatings described in cop ending applications: Ser. No. 11/096,606,filed Apr. 1, 2005, entitled: “Coated Monofilame Methods for Winding,” now U.S. Pat. Publication No. 2005-0199334 A1; Ser. No. 11/149,597, filed Jun. 10, 2005, entitled: “Non-Crystalline Saliva-Soluble Coatings for Elastomeric Monofilament Dental Tapes,” now U.S. Pat. Publication No. 2005-0226820 A1; and Ser. No. 11/196,827, filed Aug. 3, 2005, entitled: “Biofilm Therapy Interproximal Devices,” now U.S. Pat. Publication No. 2006-0034782 A1.

All of the foregoing flavor-absorbing, saliva soluble coatings can contain biofilm-responsive levels of one or more substances suitable for controlling and disrupting biofilms, while also functioning as the absorbing surface for volatile flavorants post-added to the oral products of the invention.

As used herein, “coating” refers to the process of introducing a coating substance, as well as other oral care substances onto the surfaces of various dental flosses and dental tapes.

As used herein, the phrase “saliva-soluble, flavor-absorbing, crystal-free coatings” refers to those emulsions used in compression coating of dental floss and dental tapes that indicate substantial flake resistance, yet release from various dental flosses and tape of the present invention during use when exposed to saliva in the oral cavity. These coatings can include SOFT ABRASIVES® that are dispersed and not solubilized in said coatings. These SOFT ABRASIVES® remain insoluble when delivered between teeth and below the gum line during use. Additionally, saliva-soluble, crystal-free coatings preferably contain surfactants, mouth conditioners and chemotherapeutic ingredients which are released from the dental flosses and dental tapes into interproximal sites in the oral cavity. For example, see U.S. Pat. Nos. 6,609,527 and 6,575,176.

As used herein, the term “crystal-free” refers to a smooth surface on the dental flosses and dental tapes of the invention as distinguished from rough surfaces typical of crystalline coatings when observed through a 30× stereo zoom microscope. See U.S. Pat. No. 6,609,527. Generally, crystal-free coatings indicate minimum flaking. Examples of suitable crystal-free coating formulations for various dental flosses and dental tapes of the invention are detailed in the Examples and Tables below.

As used herein, the term “biofilm therapy dental devices” refers to flavor-absorbing, coated dental devices capable of absorbing volatile flavorants from a flavor reservoir contained under flavor-sealed conditions. These devices control, disrupt and physically remove biofilms, while releasing a high-impact flavor into the oral cavity. In a preferred embodiment of the invention, the coated biofilm therapy, dental devices of the invention include a SOFT ABRASIVES® overcoating that is also released during use to work with the substrate to help physically remove and/or disrupt biofilms. Working these dental devices interproximally, massages interproximal soft tissues thereby increasing blood flow.

As used herein, the term “additional adjuvants” refers to additional ingredients that can be added to the flavor-absorbing coatings to provide color, or sweetening effects, as desired. Examples of suitable sweetening agents include sorbitol, sodium cyclamate, saccharine, commercial materials such as Nutrasweet® brand of aspartame and xylitol. Citric acid or acetic acid is often utilized as a flavor modifier and is generally used in amounts of about 1.0 to about 20 percent by weight, preferably about 2.0 percent to about 15 percent by weight.

As used herein, the term “buffering ingredient” refers to substances that may also be added to the flavor-absorbing coatings in order to prevent natural degradation of the flavoring components or therapeutically active ingredients. Generally, the pH of these compositions is adjusted from about 3.5 to about 8, depending on the chemistry of the active ingredient most requiring protection. Buffering ingredients such as an alkali metal salt of a weak organic acid, for instance, sodium benzoate, sodium citrate, sodium phosphate, sodium bicarbonate or potassium tartrate is generally added in an amount of about 0.1 to about 1.0 percent by weight. Other buffering agents such as weak organic acids or salts of weak bases and strong acids such as boric acid, citric acid, ammonium chloride, etc., can also be used in similar concentrations.

As used herein, the term “stabilizers” refers to substances that are often added to the flavor-absorbing coatings for additional control, such as:
(a) sodium benzoate, sodium or potassium sorbate, methyl paraben, propylparaben and others approved for ingestion, and
(b) chemical oxidative control substances, such as ethylene-diaminetetraacetic acid, BHA, BHT, propyl gallate and similar substances approved for ingestion. Concentration levels of these stabilizers comply with industry and regulatory standards.

As used herein, the term “SOFT ABRASIVES®” defines saliva-soluble and saliva-insoluble abrasive substances added to the flavor-adsorbable, coated dental devices of the invention that are suitable for cooperating with the various dental devices of the present invention to remove, control and disrupt biofilm, tartar and stained pellicle from tooth surfaces. SOFT ABRASIVES® include: tetrasodium pyrophosphate, calcium carbonate, dicalcium phosphate, silica, glass beads, polyethylene and polypropylene particles, pumice, titanium oxide, alumina, quartz, aluminum silicate, etc., at various particle sizes suitable for use in oral care. See U.S. Pat. No. 6,575,176.

As used herein, the term “cleaners” refers to essentially all surfactants suitable for use in the oral cavity and suitable for coating various flavor-absorbing dental devices of the present invention. For purposes of the present invention, surfactants generally are excellent adsorbers of volatile flavors.

As used herein, the phrase “chemotherapeutic ingredients” refers to those substances suitable for addition to the flavor-absorbing coatings of the present invention that impart therapeutic effects to the oral cavity including antimicrobials; anti-tartar and anti-plaque substances; remineralizing, desensitizing, NSAID and antibiotic ingredients, and the like. Specific chemotherapeutic ingredients suitable for the present invention include: stannous fluoride, potassium nitrate, cetylpyridinium chloride (CPC), triclosan, metronidazole, chlorhexidine, aspirin and doxycycline.

As used herein, the phrase “crystal formation eliminating additives” is defined as those coating additives that reduce, control and/or eliminate crystal formation and enhance the substantivity of the flavor-adsorbing coating to dental devices of the invention when added to these coatings at modest levels. These include certain aliphatic, long chain, fatty alcohols having from between about 10 and 30 carbon atoms and/or various liquid surfactants such as polyethylene glycol sorbitan dialiphatic esters.

Aliphatic, long chain, fatty alcohols are suitable for the crystal-free, flavor-absorbing coatings of the present invention. These can be represented by the structural formula ROH, wherein R represents a long chain alkyl group having from 20 to 30 carbon atoms. Specific examples include:

| | | |
|---|---|---|
| 1-decanol | 1-heptadecanol | 1-pentacosanol |
| 1 undecanol | 1-octadecanol | 1-hexacosanol |
| 1-dodecanol | 1-nonadecanol | 1-heptacosanol |
| 1-tetradecanol | 1-decosanol | 1-octacosanol |
| 1-pentadecanol | 1-henticosanol | 1-nonacosanol |
| 1-hexadecanol | 1-tricosanol | 1-triacosanol |
| 1-tetracosanol, | and mixtures thereof. | |

Naturally occurring mixtures with substantial quantities of these fatty alcohols, or isomers thereof, including those chemically derived from natural sources also constitute suitable sources of aliphatic, long chain fatty alcohols for the purpose of this invention.

The long chain fatty alcohols can be purchased commercially from Stepan, Procter & Gamble and Aldrich Chemical Co. and a variety of companies processing vegetable and animal derived fatty alcohols.

Surfactants suitable for the flavor-absorbing coatings include liquid and solid surfactants, such as:
A. Liquid surfactants including: polyoxyethylene glycol sorbitan mono- and di-aliphaticm esters represented by the general formula: 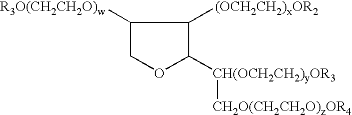
   wherein R₁, R₂, R₃, R4 and H or aliphatic acyl groups having from between about 10 and 30 carbon atoms, and the sum of w, x, y, and z is from between about 20 and about 80. These liquid surfactants are available under the trade name Emsorb®, Span®, Tween® from Cognis, N.A. and ICI. Specific example of these include: PEG 20 sorbitan monooleate (Tween® 80, ICI); PEG 40 sorbitan monostearate (SPAN 60 ICI) and PEG 40 sorbitan diisostearate (Eumulgin® SDI 40, Cognis N.A.).
B. Solid surfactants including:
   sodium lauryl sulfate,
   sodium lauryl sarcosinate,
   polyethylene glycol stearate,
   polyethylene glycol monostearate,
   coconut monoglyceride sulfonates,
   sodium alkyl sulfate,
   sodium alkyl sulfoacetates,
   block copolymers of polyoxyethylene and polyoxybutylene,
   allylpolyglycol ether carboxylates,
   polyethylene derivatives of sorbitan esters,
   propoxylated cetyl alcohol,
   block copolymers comprising a cogeneric mixtures of conjugated polyoxypropylene, and polyoxyethylene compound having as a hydrophobe a polyoxypropylene polymer of at least 1200 molecular weight (these surfactants are generally described as poloxamers; specific examples are described in the Examples below) as Poloxamer 407 and Poloxamer 388, soap powder, and mixtures thereof.

Preferably, the flavor-absorbing surfactant is included with a film forming polydimethylsiloxane, i.e., MICRODENT® or ULTRAMULSION®. As used herein, the terms “MICRODENT®” and “ULTRAMULSION®” refer to emulsion mixtures containing polydimethylsiloxane at various molecular weights in various poloxamer surfactants as described and claimed in U.S. Pat. Nos. 4,911,927; 4,950,479; 5,032,387; 5,098,711; 5,165,913; 5,538,667; 5,645,841; 5,651,959 and 5,665,374. These mouth conditioners are preferably included in crystal-free contact coatings of various dental devices of the invention.

In a particularly preferred embodiment of the invention, the flavor-absorbing coating also contains a film-forming agent. Preferably, at least one film-forming agent is utilized in the preparation of the coating mixture. Representative film-forming agents include hydroxypropyl cellulose, methyl cellulose (i.e., methyl ether of cellulose), ethyl cellulose, hydroxypropyl methyl cellulose, hydroxymethyl cellulose, carboxymethyl cellulose, gelatin, mixtures thereof, and the like. Preferably, a branched chained film-forming agent such as hydroxypropyl cellulose, is utilized. Preferably, the hydroxypropyl cellulose has a Brookfield viscosity of not less than 145 cps for a 10% aqueous solution at 25° C. The coating emulsion can contain more than one film-forming agent and as such, for example, hydroxypropyl cellulose and methyl cellulose may be utilized. In such a combination the branched chained film-forming agent (e.g., hydroxypropyl cellulose) can be used in amounts of about 0.05 to about 1.5 wt. % with about 0.01 to about 0.5 wt. % being preferred, and the straight chained film-forming agent (e.g., methyl cellulose) can be used in amounts of about 0.5 about 1.0 wt. % with about 0.1 to about 0.5 wt. % being preferred.

Another particularly preferred embodiment of the invention utilizes the unique flavor absorbing and retention properties of various dry-film forming agents and various surfactants, as described above. The modified cellulose film forming agent and the surfactants function as attractants for flavor molecules, to such an extent that they will adsorb and hold a high percentage of flavor molecules even from volatile sources after the film formers and surfactants have been coated onto the dental devices of the invention. This property of adsorbing and holding flavor molecules provides much of the consumer satisfaction associated with the dental devices of the present invention, as absorbed flavor molecules are released by the coatings upon the coatings dissolving in the saliva, thereby releasing the flavorings to olfactory organs in the oral cavity.

Volatile flavor absorption can be accomplished by coating dental devices of the invention with a suitable coating formulation minus the desired flavors, followed by a final step of introducing volatile flavoring agents from the flavor reservoir. The coated devices can then be contained in packaging which is a sufficient barrier to flavor molecules to allow the flavors to be volatilized inside the packaging without degradation, change, etc. This is easily accomplished by placing the desired quantity of volatile flavor oils into a flavor reservoir such as on a piece of absorbent paper, or into a flavor-absorbing polymer such as polyvinylacetate (Elvax® as supplied by DuPont) which releases volatile flavors at a high rate, into the purged/flavor-sealed package. Equilibrium is quickly established so that the bulk of the flavor moves from the flavor reservoir into the dry-film forming agents and/or surfactants present on the coated dental devices.

It is self-evident in the forgoing preferred embodiments that non-volatile flavor components, such as sweeteners, must be added to the initial un-flavored coatings as they cannot be transferred by equilibrium techniques.

Other substances can be added to the dental device coating including:
(a) A non-volatile flavoring agent may be present in the emulsion in an amount within the range of from about 0.1 to about 10.0 wt. %, and preferably from about 0.5 to about 3.0 wt. %, of the emulsion. The flavoring agents may comprise essential oils, synthetic flavors, or mixtures thereof including, but not limited to, oils derived from plants and fruits such as citrus oils, fruit essences, peppermint oil, spearmint oil, clove oil, oil of wintergreen, anise and the like. Artificial flavoring components are also contemplated for use in coating emulsions of the present invention. Those skilled in the art will recognize that natural and artificial flavoring agents may be combined in any sensorially acceptable blend. All such flavor sand flavor blends are contemplated by the present invention.
(b) The colorant used may include dyes, pigments, lakes and natural colors. The colorant may be blended with melted wax, preferably carnauba wax, which melts at 80°-90° C., then cooled and ground to a find particle size that will pass at least 99% through a #100 mesh sieve (less than 150 microns). Other waxes that may be used include beeswax, candelilla wax, spermaceti wax, and mixtures of the foregoing. Another method of blending is to powder blend the colorant with the wax. In either method, the preferred ratio is 1-30% colorant and 70.99% wax, and more preferably 5-15% colorant and 85-95% wax. It is preferable to use powdered colorants that have a particle size that will pass at least 99.9% through a #325 mesh sieve (small than 45 microns) so that the blended color/wax will still pass at least 99% through a #100 mesh sieve, having a particle size of 150 microns or less.
(c) Artificial sweeteners such as the soluble saccharin salts, i.e., sodium or calcium saccharin salts, cyclamate salts, acesulfam-K, and the like, and the free acid form of saccharin may optionally be added to the coating. Dipeptide sweeteners such as L-aspartyl-L-phenylalanine methyl ester and materials described in U.S. Pat. No. 3,492,131, and the like may also be used. These sweeteners may be used in amounts of about 0.005 wt. % to about 0.5 wt. % based on the weight of the total coating emulsion, and preferably about 0.05 wt. % to about 0.25 wt. %. Usually the first coating emulsion can contain about 0.02 wt. % to about 0.06 wt. % and most preferably 0.05 wt. % of artificial sweetener. A second emulsion can usually contain about 0.05 wt. % to about 0.2 wt. %, based on the weight of the coating emulsion, with about 0.08 wt. % to about 0.15 wt. % being preferred of artificial sweetener.

In a particularly preferred embodiment of the invention, the polyalcohol, xylitol, in a mixture with mono-, di- and triglycerides of the fatty acids of: palmitic, stearic and oleic acids, is included in the coating. Xylitol can be represented by the structured formula:

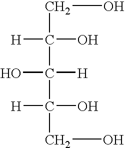

Xylitol is commercially available both in solid form and in the form of aqueous concentrated solutions. However, contrary to the saccharose shell, that of xylitol deteriorates rapidly with time. In particular, already after a few hours a xylitol shell cracks and its outer surface initially smooth becomes wrinkled; at the same time even the intimate constitution of the shell, initially sufficiently microcrystalline, changes into a course, rough structure fastidious to the palate and in chewing.

Those mixtures of glycerides are preferred which exhibit a strong prevalence of a determined glyceride. Mono- and diglycerides suitable for the purposes of this invention may present a melting point ranging from about 40° to about 70° C., keeping in mind that the melting point can be lowered (owing to formation of eutectics) by addition of a triglyceride having a convenient melting point. The preferred fatty substance is cocoa butter which, as is known, melts around 35° C.

II. Unit-Dose Packaging with Flavor-Compatible Wrapping:

Various flavor-compatible packaging materials can be used for wrapping unit-doses of the coated devices of the present invention; provided the wrapping material allows the volatile flavors present to permeate the wrapping and be absorbed by the coatings on the various dental devices free from alteration. Additionally, the wrapping must be free from flavor absorbing and/or flavor interfering glues or similar closure means. Various physical or mechanical closure means such as crimping are preferred to secure various paper wrappings around unit-doses of the coated dental flosses and dental tapes. See U.S. Pat. Nos. 2, 280,405; 3,477,191; and 6,212,860.

Suitable wrappings for the unit-dose dental devices of the present are approved by the FDA for food contact. Generally, these approved food contact papers have weights of 23 to 40 grams per square meter.

Generally, the various paper wrapping materials used to unit-dose wrap beverage straws are preferred. These include Clifford Paper soda straw wrapping paper of 23 g/m² 25 mm wide and 1.5 mil thick. Clifford Paper 0f Montreal Canada also supplies 40 g/m² pharmaceutical grade paper of 2.5-2.75 mil thick. Schweitzer-Mauduit International of Alpharetta, Ga. also provides straw wrapping paper of 25 mm wide and 22-25 g/m². See also the various Examples and Tables below.

Unexpectedly, it has been found that unit-dose packaging for flavor-adsorbable coated dental floss or dental tape can be carried out using the unit-dose packaging developed for the polypropylene beverage straws which use the Conair SWCB straw wrapping machine. That is, with slight modifications, the Conair SWCB machine can commercially wrap various coated dental devices into unit-doses of 18-22 inches in length. These paper-wrapped, unit-dose devices can then be folded, coiled, etc., and placed into a suitable flavor-sealed barrier package that is purged and provided with a flavor reservoir.

An adaptation of the SWCB straw wrapping machine to unit-dose packaging of flavor-adsorbable coated dental tapes and dental flosses is illustrated in FIG. 1 of the attached Drawings.

Referring to FIG. 1:

The SWCB machine, 1, is fitted with the Clifford Paper straw wrapping paper roll, 2, which is 25 mm wide and 7000 meters long. Dental tape, 3, is unwound from creel means, 4, and attached to the beginning folded and crimped end of paper tape, 5. As the paper is advanced by pulling of crimping wheels, 6a and 6b , tape, 3, is advanced until 22 inches is contained in paper tube.

Paper-wrapped coated dental tape is preferably converted to unit-doses and is folded offline (not shown). Paper-wrapped, coated dental tape is then folded or coiled into various shapes for packaging in a flavor-sealed package which is purged with inert gas and fitted with a flavor reservoir prior to sealing.

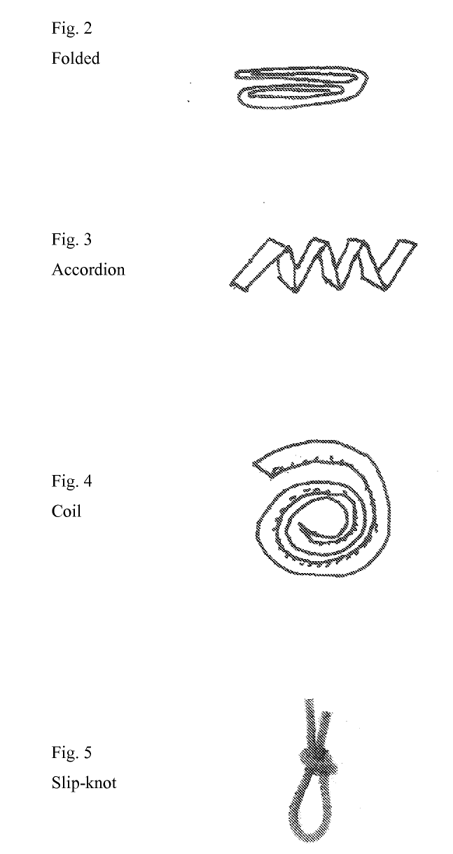

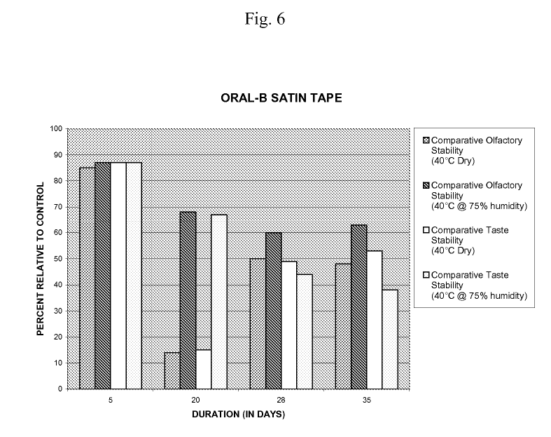

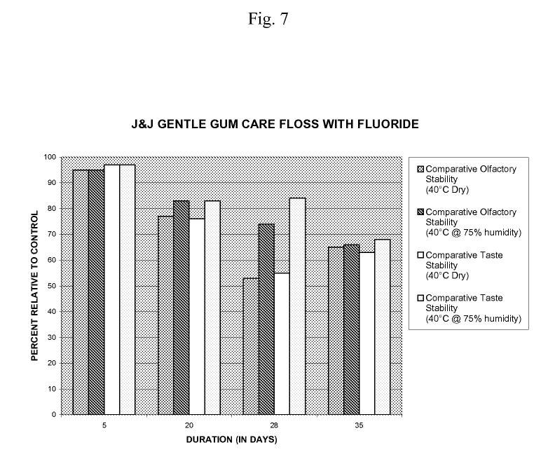

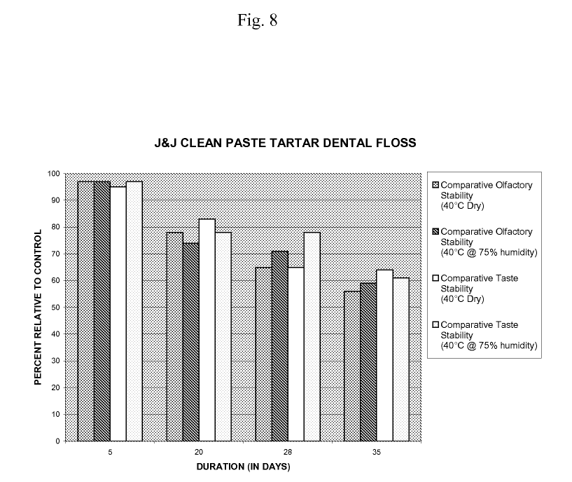

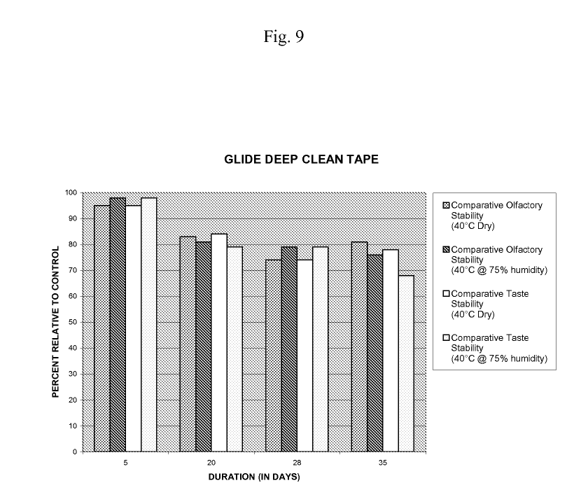

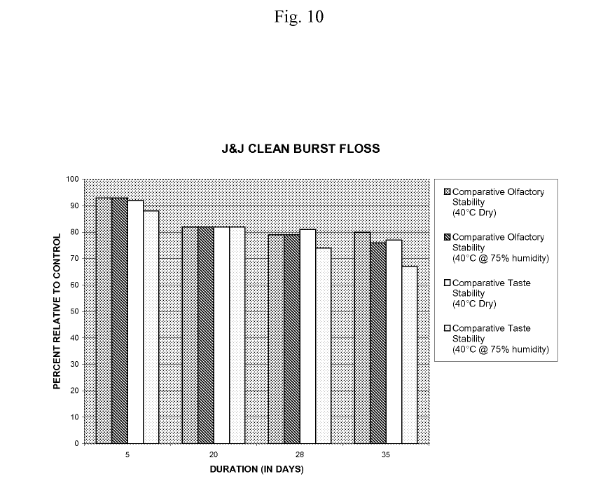

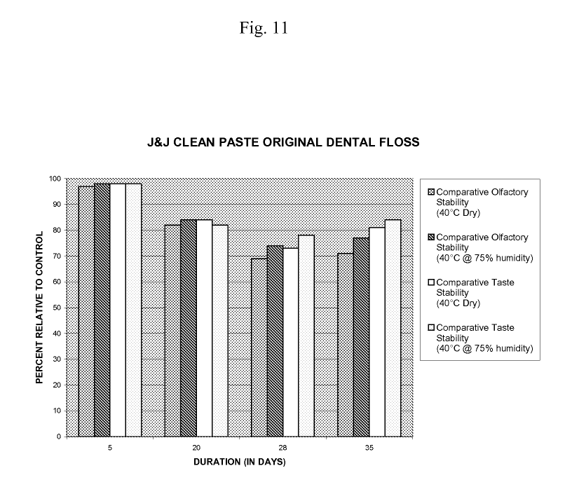

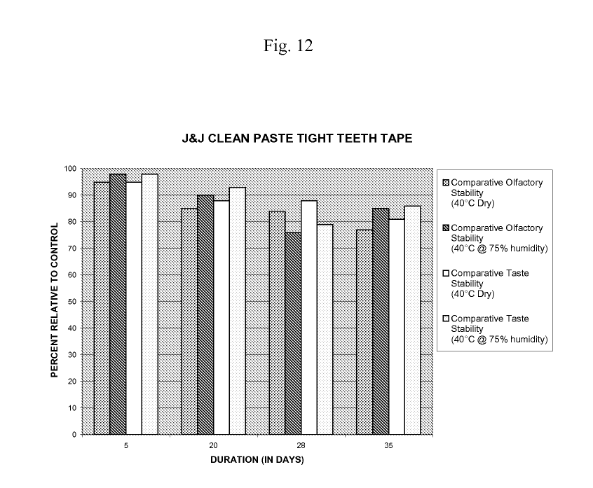

Suitable wrapping configurations for the unit-dose coated dental devices include various multi-folded, accordion-folded, coiled or slip-knotted versions such as illustrated in FIGS. 2 to 5.

These unit-dose configurations are achieved with various textile folding operations, prior to placement in flavor-sealed packages.

The “wrapped” unit-doses of coated dental flosses and dental tapes of the invention, after absorbing flavors from the flavor reservoir, are preferred over previous unit-dose versions such as described in U.S. Pat. Nos. 4,579,221; 4,693,365; 5,322,077; and 5,794,776.

The prior art, unit-dose packaging relies on various “stuffing” methods for transferring the coated dental floss or coated dental tape segment into a formed unit-dose package. Such “stuffing” operations tend to remove and/or loosen substantial quantities of the coatings and overcoatings, which is indicated by “flaking”, while also causing the monofilament dental tapes to “kink”.

Most importantly, the prior art, unit-dose packages are not flavor-sealed and thereby: (a) lose volatile top notes, (b) allow air to enter the package, and (c) allow air to react with the flavors and/or active ingredients, and/or support flavor and/or chemotherapeutic ingredient degradation during storage.

In contrast, the flavor-compatible paper wrapping process (adapted straw wrapping) of the present invention experiences minimal loss of device coatings and/or overcoatings with no kinking of monofilament substrates. Most importantly, the flavor-compatible paper wrapping allows the volatile flavors from the flavor reservoir to penetrate the paper wrap unaltered, and to be absorbed by the device coating without any alteration of the flavor. This is detailed in the Examples and Tables below.

III. Flavor-Sealed Dispenser:

Multiples of the paper-wrapped unit-doses of flavor-adsorbing coated dental tape and/or dental floss of the invention are flavor sealed in a dispenser that has been purged with an inert gas and provided with a flavor reservoir.

The flavor-sealed dispenser of the present invention serves several functions, including:

1. Providing an environment for effective transfer of volatile flavor contained in the flavor reservoir, without changes through the paper wrap, to the coating on the paper-wrapped dental devices,

2. Maintaining stability of the volatile flavor by preventing oxygen from entering the flavor-sealed dispenser during storage and by purging the dispenser with an inert gas that replaces the air (oxygen) prior to sealing,

3. Maintaining stability of certain chemotherapeutic ingredients in the device coating, assuring efficacy when these active ingredients are delivered to specific interproximal sites during use,

4. Providing long-term flavor stability to the unit-dose dental devices contained in the dispenser,

5. Dispensing coated and/or coated/overcoated dental devices with a minimum of coating/overcoating loss, while minimizing adverse physical effects on the device substrate (including freedom from kinking, fibrillating, fraying, etc.),

6. Obviating the need for plastic dispensers, bobbin winding, cutters, dispenser assembly, etc., required by bobbin-dispensed devices, and

7. Consistently dispensing high-flavor impact, coated, dental devices with stable chemotherapeutic ingredients.

The flavor-sealed dispenser provides an:

(1) excellent gas barrier,
(2) chemical resistance, and
(3) extended shelf-life to the contained, paper-wrapped unit-dose, coated dental devices of the invention.
   Preferably, various sized, shaped and designed flavor-sealed dispensers are produced from various Barex® resins that are:
(1) extruded by film and sheet extrusion, thermoformed or extrusion blow-molded,

(2) injection molded, injection blow-molded or injection stretch molded, or

(3) laminated or calenderized into sheets or film.

These Barex® resins are generally described as impact-modified acrylonitrile-methylacrylatecopolymers approved for use in food contac include:

Barex 210, Extrusion Grade,
Barex 210, Injection Grade,
Barex 210, Film Grade,
Barex 218, Extrusion Grade with increased flexibility and higher impact resistance than Barex 210, and
Barex 214 Calendar Grade.

Various Barex® films can be converted into dispenser: pouches, packages, containers, etc., suitable for flavor-sealing unit-dose, wrapped, coated dental devices using a wide range of commercial film-form-and-seal equipment such as Prodo-Pak Corporation's H-140 vertical sealer, Synchropack's PACK 900 horizontal sealer, etc.

The invention is described further but not limited by the Examples and Tables set out below.

In the Examples set below, various multifilament and monofilament dental devices of the invention are described, which are coated with a wide range of flavor-absorbing, saliva soluble coatings at varying levels and subsequently wrapped at various sizes into unit-doses using a wide range of flavor-compatible paper wrappings with flavor-compatible, securing means. Multiples of these coated unit-doses are placed into flavor-sealable dispensers fitted with a flavor reservoir and purged with an inert gas, prior to flavor-sealing.

TABLE 9
Coated Multifilament Flosses

| | | | Flavor- | | | | |
|---|---|---|---|---|---|---|---|
| | | | Compatible | | | | |
| | | | Unit-Dose | | | | |
| | | | Wrapping | | | | |
| | Type of | Saliva Soluble | Type | Inert | | Flavor Seal | |
| Ex. | Multifilament | Coating | (size in | Gas | Flavor | (type of | |
| No. | (Denier) | (mg/yd) | inches) | Purge | Reservoir | packaging) | Stability |
| 1 | Nylon 6-6 | MICRODENT ® | Soda straw | N₂ | Blotter | Heat-sealed | Long- |
| | (490) | emulsion, | paper with | | paper | (Barex ® | term |
| | | compression | crimped | | with | pouch) | |
| | | coated | seal | | Lime | | |
| | | (40) | (18-22) | | | | |
| 2 | Nylon 6-6 | MICRODENT ® | Cigarette | N₂ | Lemon | Heat-sealed | Long- |
| | (780) | emulsion with | paper with | | on edge | (Barex ® | term |
| | | Baking Soda, | crimped | | of | 2 × 3 inch | |
| | | compression | seal | | wrapping | bag) | |
| | | coated | (18) | | paper | | |
| | | (90) | | | | | |
| 3 | Polypropylene | PEG | Soda straw | N₂ | Citrus in | Mechanical- | Long- |
| | (510) | Applied by | paper with | | EVA | sealed | term |
| | | wax-coater | crimped | | resin | (Barex ® | |
| | | (35) | seal | | | heat co- | |
| | | | (20) | | | extruded | |
| | | | | | | container) | |
| 4 | PTFE | PEG | cigarette | N₂ | Mint | Heat-sealed | Long- |
| | (850) | (20) | paper | | | (Barex ® | term |
| | | | (18) | | | Envelope) | |
| 5 | PTFE | Compression | soda straw | He | Cellulose | Heat-sealed | Long- |
| | (1200) | coated | paper | | tube | (Barex ® | term |
| | | (100) | (22) | | | pouch) | |

TABLE 10
Coated Monofilament Flosses

| | | | Unit- | | | | |
|---|---|---|---|---|---|---|---|
| | | | Dose | | | | |
| | | | Wrapping | | | Flavor | |
| | Type of | Saliva Soluble | Type | Inert | | Seal | |
| Ex. | Monofilament | Coating | (size in | Gas | Flavor | (type of | |
| No. | (Denier) | (mg/yd) | inches) | Purge | Reservoir | packaging) | Stability |
| 5 | Ultra high | MICRODENT ® | Soda | Ar | Cinnamon | Heat- | Long- |
| | molecular | emulsion | straw | | | sealed | term |
| | weight | (120) | paper | | | (Barex ® | |
| | polyethylene, | | with | | | laminate | |
| | fibrillated | | crimped | | | extruded | |
| | (700) | | seal | | | envelope) | |
| | | | (20) | | | | |
| 6 | PTFE | PEG | cigarette | N₂ | Citrus | Heat- | Long- |
| | (780) | (30) | paper | | | sealed | term |
| | | | with | | | (Barex ® | |
| | | | crimped | | | extruded | |
| | | | seal | | | envelope) | |
| | | | (20) | | | | |
| 7 | High density | MICRODENT ® | Soda | N₂ | Lime | Heat- | Long- |
| | polyethylene, | (80) | straw | | | sealed | tem |
| | blow molded, | | paper | | | (Barex ® | |
| | special film | | with | | | pouch) | |
| | (540) | | crimped | | | | |
| | | | seal | | | | |
| | | | (22) | | | | |

What is claimed is:

1. A flavor-stable, dental device having a flavor-absorbing, saliva soluble coating, covered with a flavor-compatible, unit-dose packaging and contained in a flavor-sealed dispenser that has been purged with an inert gas and fitted with a volatile flavor reservoir.

2. A method for improving the long-term flavor stability of dental devices comprising:
(a) coating said devices with flavor-absorbing coating,
(b) unit-dose packaging said coated dental devices with a flavor-compatible wrapping, and
(c) storing said coated unit-dose dental devices in a flavor-sealed package that has been purged with an inert gas and provided with a volatile flavor reservoir.

3. A flavor-stable, dental device according to claim 1, wherein the flavor-absorbing, saliva soluble coating is selected from the group consisting of: surfactants, waxes, emulsions, film-forming agents, crystal-free coatings, lipowaxes, substantive coatings and combinations thereof.

4. A method according to claim 2, wherein said flavor-absorbing coating is selected from the group consisting of: surfactants, waxes, emulsions, film-forming agents, crystal-free coatings, lipowaxes, substantive coating and combinations thereof.

5. A flavor-stable, dental device according to claim 1, wherein said saliva soluble coating contains an emulsion comprising a polydimethylsiloxane dispersed in a surfactant.

6. A flavor-stable, dental device according to claim 1, wherein said saliva soluble coating contains a lipowax.

7. A flavor-stable, dental device according to claim 1, wherein said dental device, having a flavor-absorbing, saliva soluble coating, is selected from the group consisting of: nylon, multifilament dental floss; texturized nylon, multifilament dental floss; polytetrafluoroethylene, monofilament dental tape; high density polyethylene, monofilament dental tape; ultra high molecular weight, polyethylene, monofilament dental tape; and combinations thereof.

8. A method according to claim 2, wherein said dental device, having a flavor-absorbing, saliva soluble coating, is selected from the group consisting of: nylon, multifilament dental floss; texturized nylon, multifilament dental floss; polytetrafluoroethylene, monofilament dental tape; high density polyethylene, monofilament dental tape; ultra high molecular weight, polyethylene, monofilament dental tape; and combinations thereof.

9. A flavor-stable, dental device according to claim 1, wherein said saliva soluble coating contains a wax.

10. A flavor-stable, dental device according to claim 1, wherein said saliva soluble coating contains a chemotherapeutic ingredient selected from the group consisting of: stannous fluoride, baking soda, peroxide salts, hexametaphosphate, complexing ingredients, antimicrobials, antibiotics, enzymes, NSAIDs, MMP inhibitors, vitamins, nutraceuticals and combinations thereof.

11. A flavor-stable, dental device according to claim 1, wherein said flavor reservoir is selected from the group consisting of: paper, blotter material, cigarette paper, tissue paper, ethylene vinyl acetate resins, natural resins and combinations thereof.

12. A method for post-adding flavor to coated dental devices, comprising:
(a) coating said dental devices with a flavor-absorbing coating, that is substantially free from flavor,
(b) unit-dose packaging said coated dental devices with a flavor-compatible wrapping, and
(c) storing said coated, unit-dose, dental device in a flavor-sealed package that has been purged with an inert gas and provided with a flavor reservoir prior to sealing.

13. A method for improving the long-term stability of dental devices containing chemotherapeutic ingredients, comprising:
(a) coating said dental devices with a saliva soluble coating containing an active ingredient,
(b) unit-dose packaging said coated dental devices with a flavor-compatible wrapping, and
(c) storing said coated, unit-dose, dental devices in a hermetically-sealed package that has been purged with an inert gas prior to sealing.

14. A flavor-stable, dental device according to claim 1, wherein said flavor-absorbing, saliva soluble coating contains at least one sialagogue.

15. A flavor-stable, dental device according to claim 1, wherein the primary source of flavor is added to said dental device after coating.

16. An improved method for unit-dose packaging of coated dental devices, comprising wrapping said devices with a flavor-compatible, yet flavor-permeable paper that is physically secured around said coated devices in the absence of a flavor-altering means.

17. A method according to Claim 13, wherein said chemotherapeutic ingredients are selected from the group consisting of stannous fluoride, baking soda, peroxide salts, hexametaphosphate, complexing ingredients, antimicrobials, antibiotics, enzymes, NSAIDs, MMP inhibitors, vitamins, nutraceuticals and combinations thereof.

18. A method according to claim 17, wherein said chemotherapeutic ingredient is baking soda.

19. A method according to claim 17, wherein said chemotherapeutic ingredient is hexametaphosphate.

20. A flavor-stable, dental device according to claim 10, wherein said saliva soluble coating contains the chemotherapeutic ingredient, baking soda.

21. A flavor-stable, dental device according to claim 10, wherein said saliva soluble coating contains the chemotherapeutic ingredient, hexametaphosphate.

22. The dental devices described in Tables 1 through 8, converted to unit-doses, covered with a flavor-compatible packaging and contained in a flavor-sealed dispenser that has been purged with an inert gas and fitted with a volatile flavor reservoir.
